(19) United States
(12) Patent Application Publication
(10) Pub. No.: US 2015/0208994 A1
(43) Pub. Date: Jul. 30, 2015

(54) CT/MRI INTEGRATED SYSTEM FOR THE DIAGNOSIS OF ACUTE STROKES AND METHODS THEREOF

(71) Applicant: ASPECT IMAGING LTD., Shoham (IL)

(72) Inventor: Uri RAPOPORT, Moshav Ben Shemen (IL)

(21) Appl. No.: 14/598,517

(22) Filed: Jan. 16, 2015

Related U.S. Application Data
(60) Provisional application No. 61/931,706, filed on Jan. 27, 2014.

(51) Int. Cl. A61B 6/00; A61N 5/10; A61B 6/04; A61B 8/13; A61B 6/03; A61B 5/055

(52) U.S. Cl. CPC A61B 6/4417; A61B 6/032; A61B 5/0555; A61B 6/0421; A61B 6/508; A61B 6/501; A61B 8/13; A61B 6/037; A61N 5/1039; A61B 6/5247

(57) ABSTRACT

A dual modality CT/MRI integrated system for the diagnosis of acute strokes and treating a patient and methods thereof. The method includes: immobilizing a patient or a portion thereof to a support; accommodating the patient or portion thereof in a CT device; scanning the same; placing the patient or portion thereof within an MRI device and imaging the same such that the orientation of the patient or portion thereof in the CT scanning and the MRI imaging is identical or at least spatially (2D or 3D) retrievable.

### FIELD AND BACKGROUND OF THE INVENTION

The present invention, in some embodiments thereof, relates to a dual modality CT/MRI integrated system for the diagnosis of acute strokes and treating a patient and to methods thereof.

A cerebrovascular accident is the medical term for a stroke. A stroke is when blood flow to a part of the patient brain is stopped either by a blockage or a rupture of a blood vessel. The more quickly the patient gets treatment, the better the prognosis. When a stroke goes untreated for too long, there can be permanent brain damage.

There are two main types of cerebrovascular accident, or stroke. An ischemic stroke is caused by a blockage, and a hemorrhagic stroke is caused by a breakage in a blood vessel. In both cases, part of the brain is deprived of blood and oxygen, causing the brain's cells to die.

An ischemic stroke occurs when a blood clot blocks a blood vessel, preventing blood and oxygen from getting to a part of the brain. There are two ways that this can happen. When a clot forms somewhere else in body and gets lodged in a brain blood vessel, it is called an embolic stroke. When the clot forms in the brain blood vessel, it is called a thrombotic stroke.

A hemorrhagic stroke occurs when a blood vessel ruptures, or hemorrhages, which then prevents blood from getting to part of the brain. The hemorrhage may occur in a blood vessel in the brain, or in the membrane that surrounds the brain.

Diagnosis of a cerebrovascular accident is provided by various methods, such as CT or CAT scan (Computed Tomography Scan): This is a special type of x-ray that is capable of taking pictures of the brain. In some case, dye (contrast material) is used. The CAT scan is helpful in diagnosing strokes or other abnormalities of the brain. During this test, the patient will need to lie on a X-ray table. The table then is moved into the tube like opening of the scanner. There are usually no restrictions before or after the test. The test is painless and usually takes about ½ hour. Make sure that the patient tells the nurse or doctor of any allergies because of possible reaction to the dye. Sometimes a stroke cannot be seen in the first 24 hours in a CAT scan and it may be necessary to repeat the scan the following day. MRI (Magnetic Resonance Imaging): This test uses magnetic field and radio waves (not x-rays) to provide a picture of the brain. An MRI gives great detail. During this painless test, the patient lies on a narrow bed that slides into the MRI scanner. It takes about 20-45 minutes. If he or she has any metal objects in the body, such as joint replacements and surgical clips pacemakers, inform the staff. If the patient has a pacemaker, he cannot have this test. If the patient has claustrophobia, please let the staff know. The doctor may prescribe a medication to be given before the test to help the patient relax. MRA (Magnetic Resonance Angiography): This non-invasive test is like an MRI. The MRA takes pictures of the blood vessels, especially those supplying the brain. It may show the narrowing or defects in the blood vessels which help explain the source of the stroke. Unlike Angiogram, no dye is injected or used in this procedure. Carotid Duplex: This is an ultrasound study of the carotid arteries which are located in front of the neck. A probe which sends sound waves is placed over the neck area for this test. This, non-invasive test, is the most commonly used imaging test for the carotid arteries. Transesophageal Echocardiogram (TEE): This test is a specialized type of heart examination in which sound wave (ultrasound) images from a device (transducer) placed in the esophagus behind the heart are recorded. After the throat is numbed with a local anesthetic, the transducer mounted on the tip of a lighted tube is inserted into the esophagus through the mouth. It then sends and receives waves reflected from the heart. The reflected sound waves are processed by a special computer that shows an image of the heart on a video monitor. TEE gives very high quality images of the heart that cannot be obtained via the traditional transthoracic echocardiogram. To avoid vomiting and aspiration, the patient will not be allowed to eat or drink before and immediately after the procedure. Once the procedure is done, the nurse needs to evaluate the gag and swallowing reflexes to make sure these are back before the patient will be allowed to eat or drink. Transcranial Doppler (TCD): This study examines the size of the blood vessels in the brain and the direction of blood flow. In this test the person will need to lie on a table or in bed. The technician will apply gel in front of the ears, over the eyes or on the back of the head. The patient will hear a swishing sound as the probe senses the blood flowing through the blood vessels. The test takes about 30 minutes and is painless and noninvasive. The patient will be able to resume normal activities after the test. Electroencephalogram (EEG): This test records and measures patterns of electrical impulses from the brain. Tiny electrodes are placed on the scalp and a recording is made. It is very important to stay still while the recording is being done. Brain waves slow in the area of the stroke and will help doctors determine the area of damage in the brain. However, this is not a definitive test for a stroke. Cerebral Angiogram or Arteriogram: This is a special x-ray of the blood vessels of the brain taken after injecting a dye to make the vessels more visible. The dye is injected through a catheter (a long thin tube) that is inserted into an artery (usually in the groin area). The procedure takes 30-60 minutes and is useful in determining blockage and abnormalities in the blood vessels of the brain.

EP 2344033 by Orsan Medical Technologies Ltd underlines that system for diagnosing and/or choosing therapy for acute strokes using impedance plethysmography (IPG) and/or photoplethysmography (PPG) and, more particularly, but not exclusively, to a method and system for choosing or rejecting thrombolytic therapy for acute strokes. A number of cerebral hemodynamic parameters may be clinically useful for diagnosing strokes, trauma, and other conditions that can affect the functioning of the cerebrovascular system. These parameters include regional cerebral blood volume, cerebral blood flow, cerebral perfusion pressure, mean transit time, time to peak, and intracranial pressure. Many methods that are used to measure these parameters, while giving accurate results, are not practical to use for continuous monitoring, or for initial diagnosis outside a hospital setting, because they are invasive, or because they require expensive and/or non-portable equipment. Such methods include inserting a probe into the cerebrospinal fluid or into an artery, computed tomography (CT), perfusion computed tomography (PCT), positron emission tomography (PET), magnetic resonance imaging (MRI), and transcranial Doppler ultrasound (TCD).

There are many valid stroke assessment scales, such as Prehospital Stroke Assessment Scales, Cincinnati Stroke Scale, Los Angeles Prehospital Stroke Scale (LAPSS), ABCD Score, Acute Assessment Scales, Canadian Neurological Scale (CNS), European Stroke Scale, Glasgow Coma Scale (GCS), Hemispheric Stroke Scale, Hunt & Hess Scale, Mathew Stroke Scale, NIH Stroke Scale (NIHSS), NIH Stroke Scale Booklet, NIH Stroke Scale Training, Orgogozo Stroke Scale, Oxfordshire Community Stroke Project Classification (Bamford), Scandinavian Stroke Scale, World Federation of Neurological Surgeons Grading System for Subarachnoid Hemorrhage Scale, Functional Assessment Scales, Berg Balance Scale, Lawton IADL Scale, Modified, Rankin Scale, Stroke Specific Quality of Life Measure (SS-QOL), Outcome Assessment Scales, Barthel Index, Functional Independence Measurement (FIM™), Glasgow Outcome Scale (GOS), Health Survey SF-36™, Health Survey SF-12™, Community Integration Questionnaire, Action Research Arm Test, Blessed-Dementia Scale, Blessed-Dementia, Information-Memory-Concentration Test, DSM-IV criteria for the diagnosis of vascular dementia, Hachinski Ischaemia Score, Hamilton Rating Scale for Depression, NINDS-AIREN criteria for the diagnosis of vascular dementia, Orpington Prognostic Scale, Short Orientation-Memory-Concentration Test. Each of which of those scales are incorporated herein as a reference (See currently available link: http//www.strokecenter.org/professionals/stroke-diagnosis/stroke-assessment-scales/) and is denoted individually or accumulatively as “stroke's scale”.

Stroke centers in the USA provide methodical and organized stroke care. A common goal of each stroke center is to transport, assess, diagnose, and treat each stroke patient within three hours of the onset of their symptoms. To accomplish this, these specialized hospitals must have protocols in place in order to offer a well-orchestrated effort on behalf of each patient. Often, these protocols must be rehearsed and tested to ensure that the three-hour goal to treatment is accomplished.

More than that, a CT scan or MRI scanner must be available 24 hours each day, and should be reserved for stroke patients within 25 minutes of being ordered; see Stroke Unit Trialists' Collaboration. Organized inpatient (stroke unit) care for stroke. Cochrane database of Systematic Reviews 2007, Issue 4; C. S. Kidwell, et al., Establishment of primary stroke centers: a survey of physician attitudes and hospital resources Neurology 2003; 60:1452-1456; Rajajee V et al.; Early MRI and outcomes of untreated patients with mild or improving ischemic stroke. Neurology; 2006 67(6):980-4; Ali L K, Saver J L. The ischemic stroke patient who worsens: new assessment and management approaches. Rev. Neurol. Dis. 2007 Spring; 4(2):85-91; all are incorporated herein as a reference.

A CT/MRI integrated system for immediate (i.e., less than 25 minutes) diagnosis of acute strokes which provides BOTH CT and MRI diagnosis is still a long unmet need.

### BRIEF DESCRIPTION OF DRAWINGS

FIGS. 1a, 1b and 1c, schematically illustrating an embodiment of the invention in which a two-step method is illustrated. The method comprising steps of (a) immobilizing (301) a patient's (2) head (1) to a gantry (300); and then either (i) (b) accommodating said patient in a CT device (200) and scanning the same and then (c) placing said patient head within an MRI device (MRD, 100) and imaging the same such that the orientation of said head in said CT scanning and said MRI imaging is identical or at least retrievable because of said immobilization; or (ii) (b) placing said patient head within an MRD (100) and imaging the same and then accommodating said patient in a CT device (200) and scanning the same in the same manner, and then (d), superimposing said CT scans and said MRI images thereby diagnosing stroke in said patient.

FIG. 2 schematically illustrates the supporting gantry (300) with an head (1) immobilizer (301) which may comprise an RF coil for the MRD (100).

FIG. 3 schematically illustrates another embodiment of the invention, where a swivelable, rotatable, movable or reciprocating actuatable gantry is utilized.

FIG. 4 schematically illustrates an X-ray collimator (400) for emitting X-rays (401) from an orifice (402) to a field of view (target plane or volume, 403).

FIG. 5 schematically presents prior art, as disclosed in U.S. Pat. No. 8,461,841 “Means and method for thermoregulating magnets within magnetic resonance devices” by Aspect Imaging Ltd, which is incorporated herein as a reference.

FIG. 6 schematically illustrating, in a non-limiting and out of scale manner, an open bore a dual modality CT-MRD device comprising at least one opening (103), and a plurality of permanent magnets (See 140 for example). An X-ray collimator (400) (or CT collimator or PET/US effector) is locatable within the opening thus allowing scanning of head (1) within the open bore (150) by the X-Ray (403) whilst MR imaging the same.

FIGS. 7a and 7b schematically illustrating a dual modality X-ray/MRI device for image-guided radiation therapy of a patient. In FIG. 7a, two X-ray collimators (420, 410 for example) are either temporarily or not affixed within opening at opposite directions at each side wall of the device. Collimated X-rays (422 and 411, respectively) are emitted over an object, here e.g., patient head (1) having a tumor to analyze and treat. 422 and 411 are emitted in parallel yet opposite directions. In FIG. 7b, however, the two X-ray collimators (420, 410 for example) are either temporarily or not affixed within opening at opposite directions at each side wall of the device. Collimated X-rays (422 and 411, respectively) are emitted over the object, here again, e.g., patient head (1) having a tumor to analyze and treat. 422 and 411 are converges at a predefined angle.

FIGS. 8a-8c schematically illustrating a continuous two-steps method of diagnosing a person. At the first step, the patient (2) is supported on linearly reciprocating gantry (360) made of MRI-safe materials, and scanned throughout a first diagnosing device (e.g., CT 200, MRI etc.), see whole-body scan in FIG. 8b. Then the patient, still on said gantry 360, is further advanced to a neighboring adjacent second device, such as a commercially available magnetic-non-fringing device by Aspect Imaging Ltd (US) open bore permanent magnet head-MRI 100, CT, PET, a PET-MRI dual modality MRD as defined below, etc. Only the head (1) is inserted within said MRI device in this example (See FIG. 8c).

FIG. 8d schematically discloses another embodiment of the invention, namely a conjugated (linearly integrated) CT/MRI dual modality device or MRICT dual modality device where the patient is inserted (whole body) in one device (e.g., CT 200) whilst his head further accommodated within the open-bore of a second device (e.g., MRI 100, such as a commercially available M-type MRI by Aspect Imaging Ltd (US)) and vice versa.

### SUMMARY OF THE INVENTION

It is thus an object of the present invention to disclose a two step method of diagnosing a medical condition in a patient, wherein said method comprising steps of (a) immobilizing (301) a patient's (2) or a portion thereof (1) to a support (300); and then either (i) (b) accommodating said patient or a portion thereof in a CT device (200) and scanning the same and then (c) placing said patient or portion thereof within an MRI device (MRD, 100) and imaging the same such that the orientation of said patient or portion thereof in said CT scanning and said MRI imaging is identical or at least spatially (2D or 3D) retrievable; or (ii) (b) placing said patient or portion thereof within an MRD (100) and imaging the same and then accommodating said patient or portion thereof in a CT device (200) and scanning the same in the same manner, and then (d), superimposing or otherwise correlating between at least a portion of said CT scans and said MRI images thereby diagnosing the medical condition of said patient or portion thereof.

It is another object of the present invention to disclose a two step method of diagnosing stroke in a patient, wherein said method comprising steps of (a) immobilizing (301) a patient's (2) head (1) to a gantry (300); and then either (i) (b) accommodating said patient in a CT device (200) and scanning the same and then (c) placing said patient head within an MRI device (MRD, 100) and imaging the same such that the orientation of said head in said CT scanning and said MRI imaging is identical or at least retrievable because of said immobilization; or (ii) (b) placing said patient head within an MRD (100) and imaging the same and then accommodating said patient in a CT device (200) and scanning the same in the same manner; and then (d), superimposing said CT scans and said MRI images thereby diagnosing stroke in said patient.

It is another object of the present invention to disclose a two step method of diagnosing stroke in a patient by means of rotatable or otherwise movable gantry; wherein said method comprising steps of (a) providing a rotatable (302) or otherwise movable gantry; (b) immobilizing (301) a patient's (2) or portion thereof (1) to said gantry (300); and then either (i), (c) providing said gantry in a first configuration, and accommodating said patient in a CT device (200) and scanning the same; and then (d rotating (303) or otherwise moving (304) the gantry to a second configuration, and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said patient in a CT device (200) and scanning the same in the same manner, and then (e), superimposing or otherwise correlating between at least a portion of said CT scans and said MRI images thereby diagnosing the medical condition of said patient or portion thereof.

It is another object of the present invention to disclose a single step method of diagnosing an object; wherein said method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI) effector within said at least one first opening facing said proximal aperture towards said open bore; (c) accommodating an object to image within said open bore (the FOV); (d) either concurrently or subsequently diagnosing said object whilst or before/after MR imaging the same, respectively, thereby providing superimposed or spatially correlated MRI/NMRI diagnosis of said object.

It is another object of the present invention to disclose a single step method of diagnosing an object; wherein said method comprising steps of (a) providing an open-bore MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI) effector within said at least one first opening facing said proximal aperture towards said open bore; (c) providing at least one second non-MRI diagnosing (2NMRI) effector within said at least one second opening facing said proximal aperture towards said open bore; (d) accommodating an object to image within said open bore (the FOV); (e) concurrently and/or subsequently diagnosing said object by means of said 1NMRI and 2NMRI devices whilst or before/after MR imaging the same, respectively, thereby providing superimposed or spatially correlated MRI/1NMRI/2NMRI diagnosis of said object.

It is another object of the present invention to disclose a single step method of diagnosing a stroke in a patient; wherein said method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said patient or a portion thereof within said open bore (the FOV); (d) either concurrently or subsequently diagnosing said object whilst or before/after MR imaging the same, respectively, thereby providing superimposed or spatially correlated MRI/NMRI image of said patient or portion thereof.

It is another object of the present invention to disclose a single step method of diagnosing a stroke in a patient; wherein said method comprising steps of (a) providing an open-bore MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI such as one device selected from the following X-ray, CT, PET, or a ultra sound device) effector within said at least one first opening facing said proximal aperture towards said open bore; (c) providing at least one second non-MRI diagnosing (2NMRI, such as another device selected from the following X-ray, CT, PET, or a ultra sound device) effector within said at least one second opening facing said proximal aperture towards said open bore; (d) accommodating an object to image within said open bore (the FOV); (e) concurrently and/or subsequently diagnosing said object by means of said 1NMRI and 2NMRI devices whilst or before/after MR imaging the same, respectively, thereby providing superimposed or spatially correlated MRI/1NMRI/2NMRI and diagnosing a stroke in a patient.

It is another object of the present invention to disclose a two step method of diagnosing stroke in a patient, wherein said method comprises steps of (a) immobilizing (301) a patient's (2) head (1) to a gantry (300); and then either (i) (b) accommodating said patient in a CT device (200) and scanning the same and then (c) placing said patient head within an MRI device (MRD, 100) and imaging the same such that the orientation of said head in said CT scanning and said MRI imaging is identical or at least retrievable because of said immobilization; or (ii) (b) placing said patient head within an MRD (100) and imaging the same and then accommodating said patient in a CT device (200) and scanning the same in the same manner; and then (d), superimposing said CT scans and said MRI images thereby analyzing stroke in said patient.

It is another object of the present invention to disclose a rotatable or otherwise movable gantry for diagnosing an object wherein said gantry is operative in a method comprising steps of (a) providing said gantry with a rotating means (302) or moving mechanism; (b) immobilizing (301) a patient's (2) or portion thereof (1) to said gantry (300); and then either (i), (c) providing said gantry in a first configuration, and accommodating said object in a CT device (200) and scanning the same; and then (d rotating (303) or otherwise moving (304) the gantry to a second configuration, and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said object in a CT device (200) and scanning the same in the same manner, and then (e), superimposing or otherwise correlating between at least a portion of said CT scans and said MRI images thereby analyzing said object

It is another object of the present invention to disclose a rotatable or otherwise movable gantry for diagnosing stroke in a patient wherein said gantry is operative in a method comprising steps of (a) providing said gantry with a rotating means (302) or moving mechanism; (b) immobilizing (301) a patient's (2) or portion thereof (1) to said gantry (300); and then either (i), (c) providing said gantry in a first configuration, and accommodating said patient in a CT device (200) and scanning the same; and then (d rotating (303) or otherwise moving (304) the gantry to a second configuration, and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said patient in a CT device (200) and scanning the same in the same manner, and then (e), superimposing or otherwise correlating between at least a portion of said CT scans and said MRI images thereby diagnosing the medical condition of said patient or portion thereof.

It is another object of the present invention to disclose an open-bore MRI device for diagnosing an object; wherein said MRI is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

It is another object of the present invention to disclose an open-bore MRI device for diagnosing an object; wherein MRD is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

It is another object of the present invention to disclose an open-bore MRI device for diagnosing a stroke in a patient; wherein said MRI is operative in method comprising steps of (a) providing an open-bore permanent magnet MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI such as one device selected from the following X-ray, CT, PET, or an ultra sound device) effector within said at least one first opening facing said proximal aperture towards said open bore; (c) providing at least one second non-MRI diagnosing (2NMRI, such as another device selected from the following X-ray, CT, PET, or an ultra sound device) effector within said at least one second opening facing said proximal aperture towards said open bore; (d) accommodating an object to image within said open bore (the FOV); (e) concurrently and/or subsequently diagnosing said patient or portion thereof by means of said 1NMRI and 2NMRI devices whilst or before/after MR imaging the same, respectively, thereby providing superimposed or spatially correlated MRI/1NMRI/2NMRI and diagnosing a stroke in said patient.

It is another object of the present invention to disclose an open-bore MRI device for diagnosing an object; wherein MRD is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

It is another object of the present invention to disclose an open-bore MRI device for diagnosing a stroke in a patient; wherein said MRD comprising at least two permanent magnets (140); at least two openings (103a, 103b) each of which having a proximal aperture within said MRI open bore (e.g., field of view) and distal aperture at said MRD outside shell; at least one first non-MRI diagnosing means (1NMRI such as one device selected from the following X-ray, CT, PET, or an ultra sound device) locatable within said at least one first opening facing said proximal aperture towards said open bore; and optionally at least one second non-MRI diagnosing (2NMRI, such as another device selected from the following X-ray, CT, PET, or an ultra sound device) locatable within said at least one second opening facing said proximal aperture towards said open bore.

It is another object of the present invention to disclose the object or patient as defined in any of the above, wherein it is selected from human patients, neonates, premature babies, laboratory animals and portions thereof (head, liver etc), histology preparations and plants.

### DESCRIPTION OF EMBODIMENTS OF THE INVENTION

X-radiation (composed of X-rays) is a form of electromagnetic radiation. Most X-rays have a wavelength in the range of 0.01 to 10 nanometers, corresponding to frequencies in the range 30 petahertz to 30 exahertz (3×10¹⁶ Hz to 3×10¹⁹ Hz) and energies in the range 100 eV to 100 keV. Fluoroscopy is an imaging technique commonly used by physicians or radiation therapists to obtain real-time moving images of the internal structures of a patient through the use of a fluoroscope. In its simplest form, a fluoroscope consists of an X-ray source and fluorescent screen between which a patient is placed. However, modern fluoroscopes couple the screen to an X-ray image intensifier and CCD video camera allowing the images to be recorded and played on a monitor. This method may use a contrast material. Examples include cardiac catheterization (to examine for coronary artery blockages) and barium swallows (to examine for esophageal disorders). The use of X-rays as a treatment is known as radiation therapy and is largely used for the management (including palliation) of cancer; it requires higher radiation energies than for imaging alone. In X-ray optics, a collimator is a device that filters a stream of rays so that only those traveling parallel to a specified direction are allowed through.

Reference is now made to FIG. 4, schematically illustrating an X-ray collimator (400) for emitting X-rays (401) from an orifice (402) to a field of view (target plane or volume, 403). X-ray computed tomography (x-ray CT) is a technology that uses computer-processed x-rays to produce tomographic images (virtual ‘slices’) of specific areas of the scanned object, allowing the user to see what is inside it without cutting it open. Digital geometry processing is used to generate a three-dimensional image of the inside of an object from a large series of two-dimensional radiographic images taken around a single axis of rotation. Medical imaging is the most common application of x-ray CT. Its cross-sectional images are used for diagnostic and therapeutic purposes in various medical disciplines. CT scanning of the head is typically used to detect infarction, tumors, calcifications, haemorrhage and bone trauma. Of the above, hypodense (dark) structures can indicate edema and infarction, hyperdense (bright) structures indicate calcifications and haemorrhage and bone trauma can be seen as disjunction in bone windows. Tumors can be detected by the swelling and anatomical distortion they cause, or by surrounding edema. Ambulances equipped with small bore multi-slice CT scanners can respond to cases involving stroke or head trauma.

Anti-scatter collimators (also referred to as anti-scatter septa or grids) are used in CT X-ray equipment used for medical imaging, but also non-medical X-ray applications such as cargo scanners at airports. CT scanners consist of an x-ray source opposite an arc-shaped array of detectors. The collimator is located immediately in front of the detectors to protect them from scattered X-rays. Ideally, each detector in a CT scanner measures intensity of X-rays that reach the detector after traveling along a straight-line path from the X-ray source to the detector. The commonly known anti-scatter collimator is comprised of thin plates formed from a suitable X-ray absorbing material like lead or tungsten. These plates are focused at the x-ray focal spot and generally located between columns of detectors (z-direction) but not between rows of detectors. This collimator is referred to as a “1D” anti-scatter collimator. In multi-slice scanners it has been found more advantageous to have shielding between both columns and rows of detectors; both directions are focusing to the X-ray source. This type is called a “2D” anti-scatter collimator.

Magnetic resonance imaging (MRI) of the head provides superior information as compared to CT scans when seeking information about headache to confirm a diagnosis of neoplasm, vascular disease, posterior cranial fossa lesions, cervicomedullary lesions, or intracranial pressure disorders. It also does not carry the risks of exposing the patient to ionizing radiation. CT scans may be used to diagnose headache when neuroimaging is indicated and MRI is not available, or in emergency settings when hemorrhage, stroke, or traumatic brain injury are suspected.

Positron emission tomography (PET) is a nuclear medicine, functional imaging technique that produces a three-dimensional image of functional processes in the body. The system detects pairs of gamma rays emitted indirectly by a positron-emitting radionuclide (tracer), which is introduced into the body on a biologically active molecule. Three-dimensional images of tracer concentration within the body are then constructed by computer analysis. In modern PET-CT scanners, three dimensional imaging is often accomplished with the aid of a CT X-ray scan performed on the patient during the same session, in the same machine.

If the biologically active molecule chosen for PET is fludeoxy-glucose (FDG), an analogue of glucose, the concentrations of tracer imaged will indicate tissue metabolic activity by virtue of the regional glucose uptake. Use of this tracer to explore the possibility of cancer metastasis (i.e., spreading to other sites) is the most common type of PET scan in standard medical care (90% of current scans). However, on a minority basis, many other radioactive tracers are used in PET to image the tissue concentration of many other types of molecules of interest. PET scans are increasingly read alongside CT or magnetic resonance imaging (MRI) scans, with the combination (called “co-registration”) giving both anatomic and metabolic information (i.e., what the structure is, and what it is doing biochemically). Because PET imaging is most useful in combination with anatomical imaging, such as CT, modern PET scanners are now available with integrated high-end multi-detector-row CT scanners (so-called “PET-CT”). Because the two scans can be performed in immediate sequence during the same session, with the patient not changing position between the two types of scans, the two sets of images are more-precisely registered, so that areas of abnormality on the PET imaging can be more perfectly correlated with anatomy on the CT images.

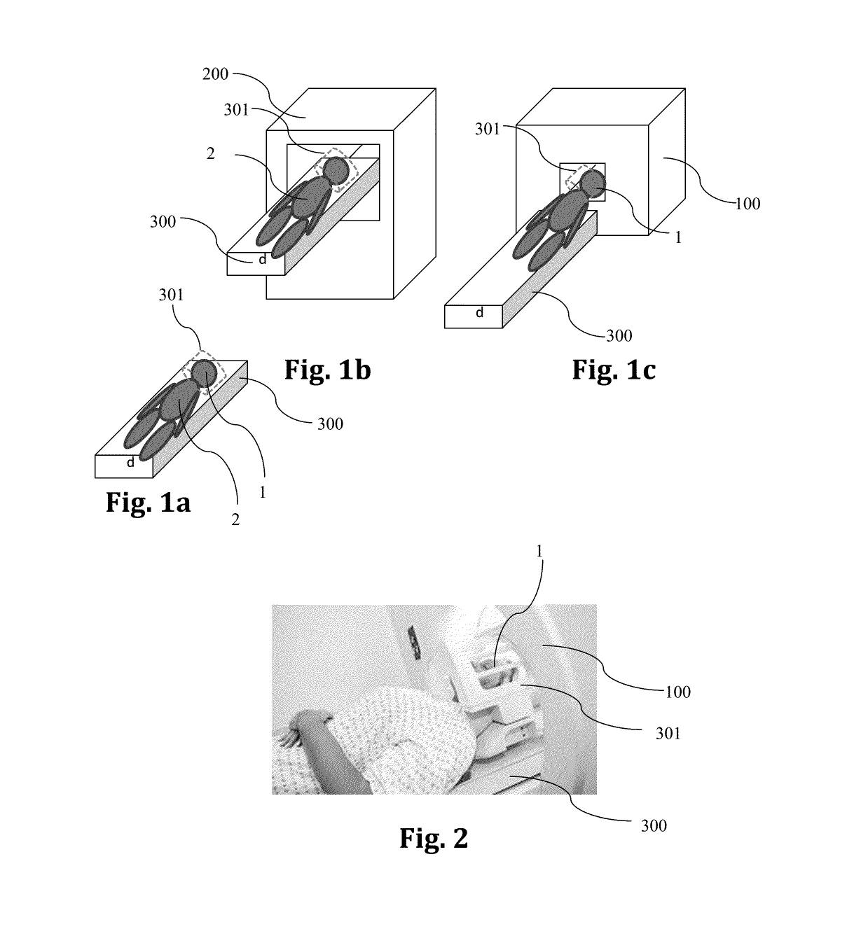

Reference is now made to FIGS. 1a, 1b and 1c, schematically illustrating an embodiment of the invention. According to said embodiment of the invention, a two step method of diagnosing a medical condition in a patient is disclosed. The method comprising steps of (a) immobilizing (301) a patient's (2) or a portion thereof (1) to a support (300); and then either (i) (b) accommodating said patient or a portion thereof in a CT device (200) and scanning the same and then (c) placing said patient or portion thereof within an MRI device (MRD, 100) and imaging the same such that the orientation of said patient or portion thereof in said CT scanning and said MRI imaging is identical or at least spatially (2D or 3D) retrievable; or (ii) (b) placing said patient or portion thereof within an MRD (100) and imaging the same and then accommodating said patient or portion thereof in a CT device (200) and scanning the same in the same manner; and then (d), superimposing or otherwise correlating between at least a portion of said CT scans and said MRI images thereby diagnosing the medical condition of said patient or portion thereof.

According to another embodiment of the invention, a two step method of diagnosing a medical condition in a patient is disclosed. Reference is now made to FIGS. 1a-c and to FIG. 2, illustrating the supporting gantry (300) with an head (1) immobilizer (301) which may comprise an RF coil for the MRD (100). The method comprising steps of (a) immobilizing (301) a patient's (2) head (1) to a gantry (300); and then either (i) (b) accommodating said patient in a CT device (200) and scanning the same and then (c) placing said patient head within an MRI device (MRD, 100) and imaging the same such that the orientation of said head in said CT scanning and said MRI imaging is identical or at least retrievable because of said immobilization; or (ii) (b) placing said patient head within an MRD (100) and imaging the same and then accommodating said patient in a CT device (200) and scanning the same in the same manner; and then (d), superimposing said CT scans and said MRI images thereby diagnosing stroke in said patient.

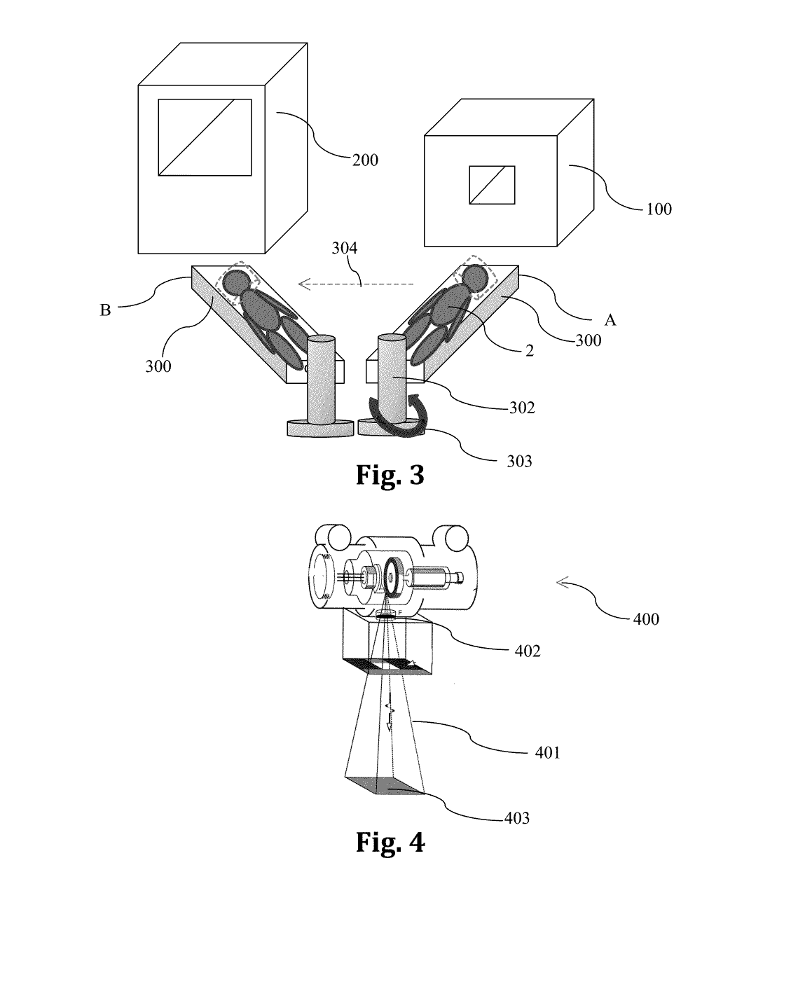

Reference is now made to FIG. 3, schematically illustrating another embodiment of the invention, where a swivelable, rotatable, movable or reciprocating actuatable gantry is utilized. A two step method of diagnosing stroke in a patient by means of rotatable or otherwise movable gantry; wherein said method comprising steps of (a) providing a rotatable (302) or otherwise movable gantry; (b) immobilizing (301) a patient's (2) or portion thereof (1) to said gantry (300); and then either (i), (c) providing said gantry in a first configuration (A), and accommodating said patient in a CT device (200) and scanning the same; and then (d rotating (303) or otherwise moving (304) the gantry to a second configuration (B), and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said patient in a CT device (200) and scanning the same in the same manner; and then (e), superimposing or otherwise correlating between at least a portion of said CT scans and said MRI images thereby diagnosing the medical condition of said patient or portion thereof.

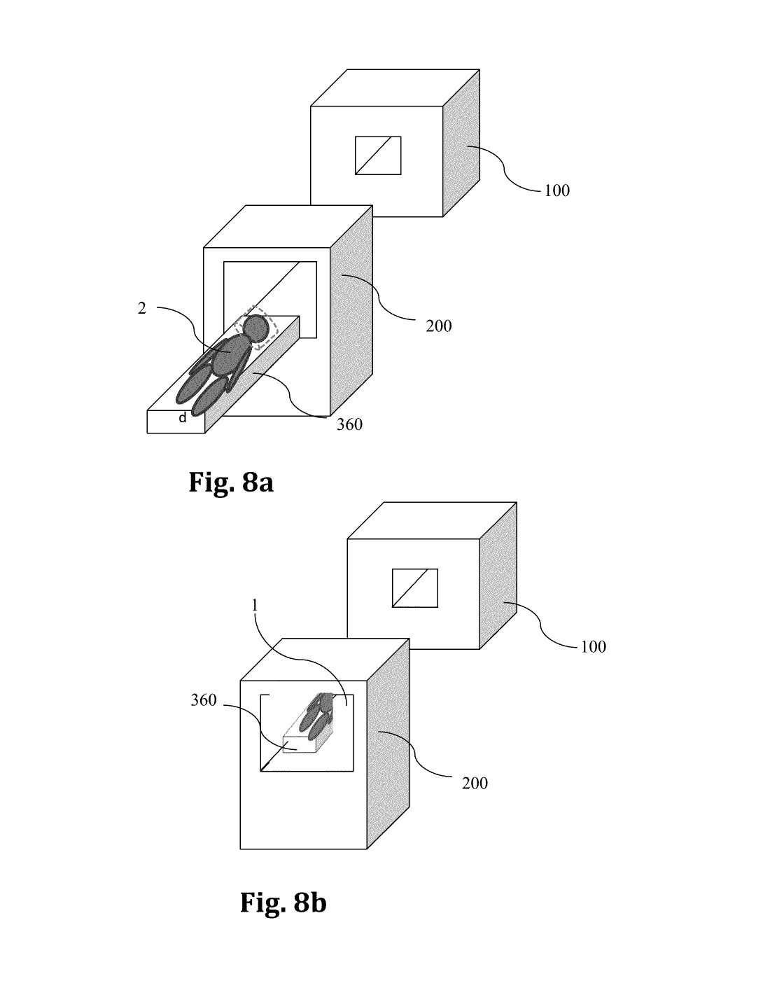

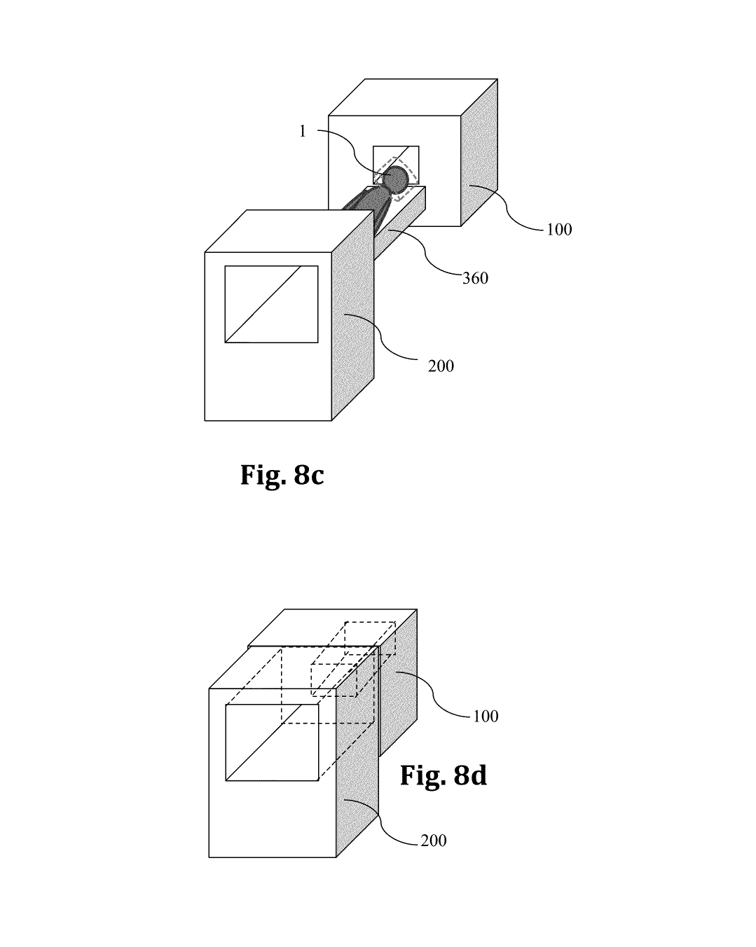

Reference is now made to FIGS. 8a-8c schematically illustrating a continuous two-steps method of diagnosing a person. At the first step, the patient (2) is supported on linearly reciprocating gantry (360) made of MRI-safe materials, and scanned throughout a first diagnosing device (e.g., CT 200, MRI etc.), see whole-body scan in FIG. 8b. Then the patient, still on said gantry 360, is further advanced to a neighboring adjacent second device, such as a commercially available magnetic-non-fringing device by Aspect Imaging Ltd (US) open bore permanent magnet head-MRI 100, CT, PET, a PET-MRI dual modality MRD as defined below, etc. Only the head (1) is inserted within said MRI device in this example (See FIG. 8c).

FIG. 8d schematically discloses another embodiment of the invention, namely a conjugated (linearly integrated) CT/MRI dual modality device or MRICT dual modality device where the patient is inserted (whole body) in one device (e.g., CT 200) whilst his head further accommodated within the open-bore of a second device (e.g., MRI 100, such as a commercially available M-type MRI by Aspect Imaging Ltd (US)) and vice versa.

A single step method of diagnosing an object; wherein said method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI) effector within said at least one first opening facing said proximal aperture towards said open bore; (c) accommodating an object to image within said open bore (the FOV); (d) either concurrently or subsequently diagnosing said object whilst or before/after MR imaging the same, respectively, thereby providing superimposed or spatially correlated MRI/NMRI diagnosis of said object.

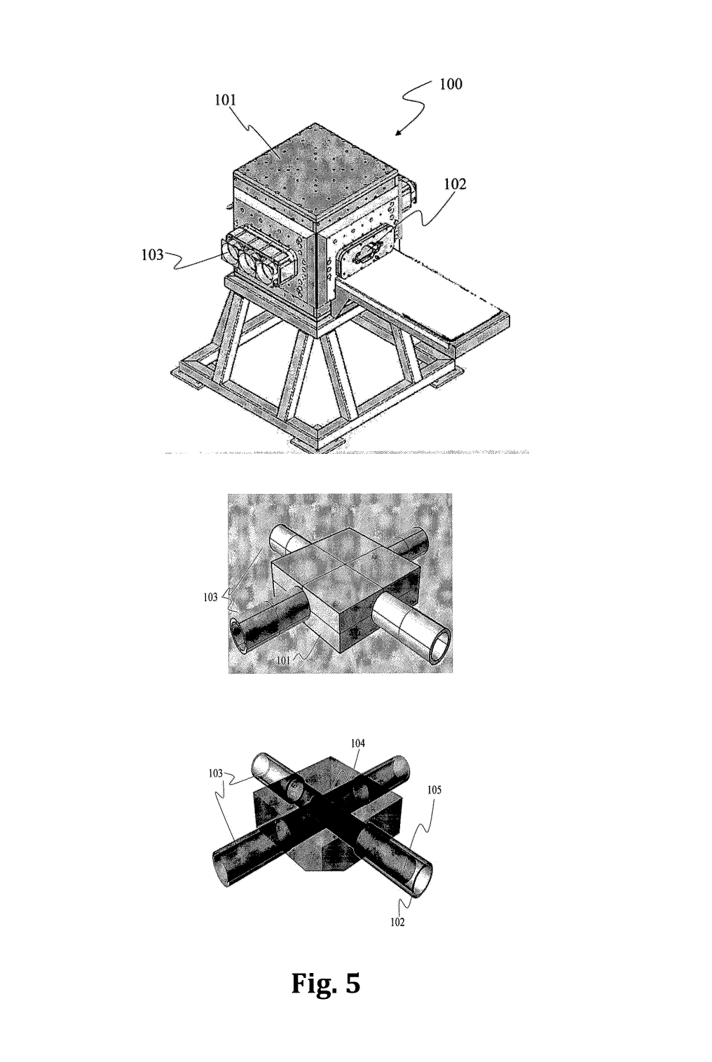

Reference is now made to FIG. 5, schematically presents prior art, as disclosed in U.S. Pat. No. 8,461,841 “Means and method for thermoregulating magnets within magnetic resonance devices” by Aspect Imaging Ltd, which is incorporated herein as a reference. This commercially available permanent magnet (101) open bore MRI device (100), by Aspect Imaging Ltd (US) comprises a plurality of openings (see 103), open bore aperture (102). FIG. 5 further illustrates cross sections of the MRD, showing the plurality of eth openings (103).

A single step method of diagnosing an object; wherein said method comprising steps of (a) providing an open-bore MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI) effector within said at least one first opening facing said proximal aperture towards said open bore; (c) providing at least one second non-MRI diagnosing (2NMRI) effector within said at least one second opening facing said proximal aperture towards said open bore; (d) accommodating an object to image within the open bore (the FOV); (e) concurrently and/or subsequently diagnosing said object by means of said 1NMRI and 2NMRI devices whilst or before/after MR imaging the same, respectively, thereby providing superimposed or spatially correlated MRI/1NMRI/2NMRI diagnosis of said object.

A single step method of diagnosing a stroke in a patient; wherein said method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said patient or a portion thereof within said open bore (the FOV); (d) either concurrently or subsequently diagnosing said object whilst or before/after MR imaging the same, respectively, thereby providing superimposed or spatially correlated MRI/NMRI image of said patient or portion thereof.

A single step method of diagnosing a stroke in a patient; wherein said method comprising steps of (a) providing an open-bore MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI such as one device selected from the following X-ray, CT, PET, or a ultra sound device) effector within said at least one first opening facing said proximal aperture towards said open bore; (c) providing at least one second non-MRI diagnosing (2NMRI, such as another device selected from the following X-ray, CT, PET, or a ultra sound device) effector within said at least one second opening facing said proximal aperture towards said open bore; (d) accommodating an object to image within said open bore (the FOV); (e) concurrently and/or subsequently diagnosing said object by means of said 1NMRI and 2NMRI devices whilst or before/after MR imaging the same, respectively, thereby providing superimposed or spatially correlated MRI/1NMRI/2NMRI and diagnosing a stroke in a patient.

A two step method of diagnosing stroke in a patient, wherein said method comprising steps of (a) immobilizing (301) a patient's (2) head (1) to a gantry (300); and then either (i) (b) accommodating said patient in a CT device (200) and scanning the same and then (c) placing said patient head within an MRI device (MRD, 100) and imaging the same such that the orientation of said head in said CT scanning and said MRI imaging is identical or at least retrievable because of said immobilization; or (ii) (b) placing said patient head within an MRD (100) and imaging the same and then accommodating said patient in a CT device (200) and scanning the same in the same manner; and then (d), superimposing said CT scans and said MRI images thereby analyzing stroke in said patient.

A rotatable or otherwise movable gantry for diagnosing an object wherein said gantry is operative in a method comprising steps of (a) providing said gantry with a rotating means (302) or moving mechanism; (b) immobilizing (301) a patient's (2) or portion thereof (1) to said gantry (300); and then either (i), (c) providing said gantry in a first configuration, and accommodating said object in a CT device (200) and scanning the same; and then (d rotating (303) or otherwise moving (304) the gantry to a second configuration, and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said object in a CT device (200) and scanning the same in the same manner; and then (e), superimposing or otherwise correlating between at least a portion of said CT scans and said MRI images thereby analyzing said object

A rotatable or otherwise movable gantry for diagnosing stroke in a patient wherein said gantry is operative in a method comprising steps of (a) providing said gantry with a rotating means (302) or moving mechanism; (b) immobilizing (301) a patient's (2) or portion thereof (1) to said gantry (300); and then either (i), (c) providing said gantry in a first configuration, and accommodating said patient in a CT device (200) and scanning the same; and then (d rotating (303) or otherwise moving (304) the gantry to a second configuration, and placing said patient or portion thereof within an MRI device (MRD, 100) and imaging the same such that the orientation of said head in said CT scanning and said MRI imaging is identical or at least spatially (2d or 3D) retrievable because of said immobilization; or (ii) (c) providing the gantry in its first configuration and placing said patient head within an MRD (100) and imaging the same; (d) rotating or otherwise moving the gantry to its second configuration and accommodating said patient in a CT device (200) and scanning the same in the same manner; and then (e), superimposing or otherwise correlating between at least a portion of said CT scans and said MRI images thereby diagnosing the medical condition of said patient or portion thereof.

An open-bore MRI device for diagnosing an object; wherein said MRI is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

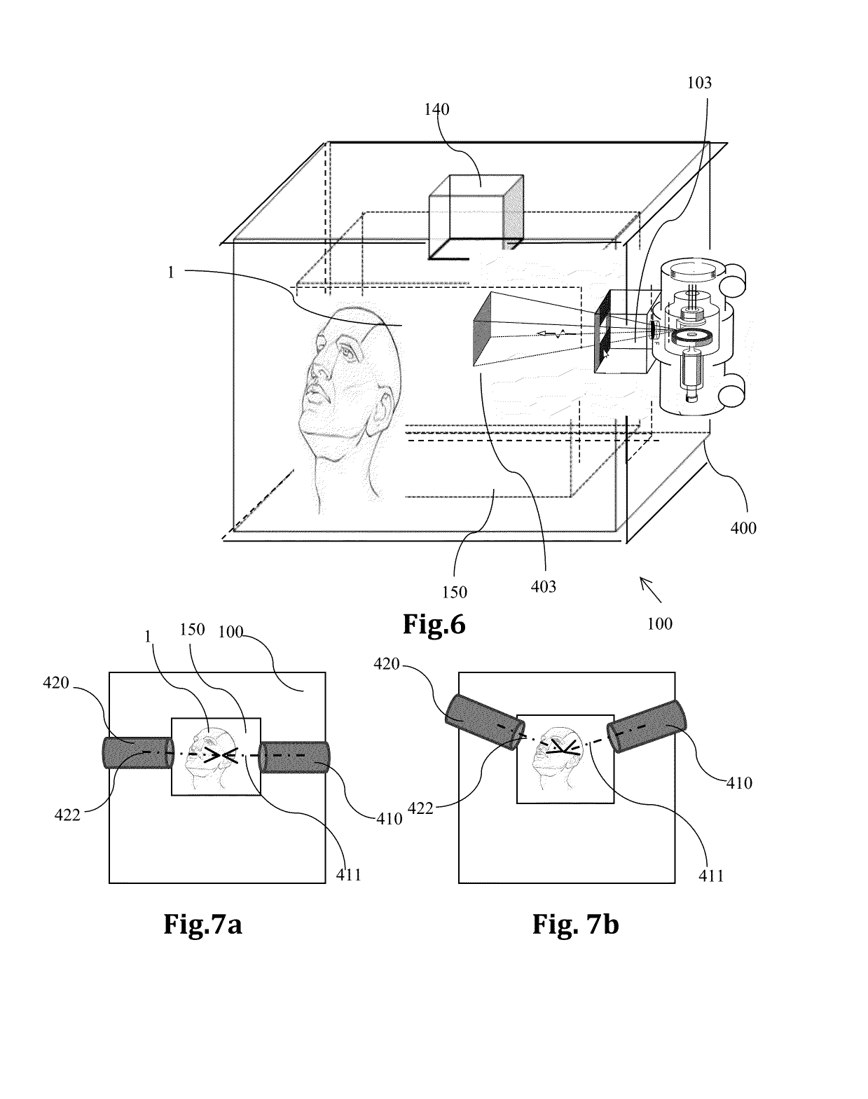

Reference is now made to FIG. 6, schematically illustrating, in a non-limiting and out of scale manner, an open bore a dual modality CT-MRD device comprising at least one opening (103), and a plurality of permanent magnets (See 140 for example). An X-ray collimator (400) (or CT collimator or PET/US effector) is locatable within the opening thus allowing scanning of head (1) within the open bore (150) by the X-Ray (403) whilst MR imaging the same.

An open-bore MRI device for diagnosing an object; wherein MRD is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

An open-bore MRI device for diagnosing a stroke in a patient; wherein said MRI is operative in method comprising steps of (a) providing an open-bore permanent magnet MRI device (MRD, 100) with at least two openings (103a, 103b) each of which having a proximal aperture within said MRI open bore (e.g., field of view) and distal aperture at said MRD outside shell; (b) providing at least one first non-MRI diagnosing (1NMRI such as one device selected from the following X-ray, CT, PET, or an ultra sound device) effector within said at least one first opening facing said proximal aperture towards said open bore; (c) providing at least one second non-MRI diagnosing (2NMRI, such as another device selected from the following X-ray, CT, PET, or an ultra sound device) effector within said at least one second opening facing said proximal aperture towards said open bore; (d) accommodating an object to image within said open bore (the FOV); (e) concurrently and/or subsequently diagnosing said patient or portion thereof by means of said 1NMRI and 2NMRI devices whilst or before/after MR imaging the same, respectively, thereby providing superimposed or spatially correlated MRI/1NMRI/2NMRI and diagnosing a stroke in said patient.

An open-bore MRI device for diagnosing an object; wherein MRD is operative in a method comprising steps of (a) providing an open-bore MRI device (MRD, 100) with at least one opening (103) having a proximal aperture within said MRI open bore (e.g., field of view) and distal aperture at said MRD outside shell; (b) providing at least one first non-MRI diagnosing (NMRI, such as X-ray, CT, PET, ultra sound devices) effector within said at least one first opening facing said proximal aperture towards said open bore; (c) accommodating said object within said open bore (the FOV); (d) either concurrently or subsequently diagnosing said object whilst or before/after MR imaging the same, respectively, thereby providing superimposed or spatially correlated MRI/NMRI image of said object.

An open-bore MRI device for diagnosing a stroke in a patient; wherein said MRD comprising at least two permanent magnets (140); at least two openings (103a, 103b) each of which having a proximal aperture within said MRI open bore (e.g., field of view) and distal aperture at said MRD outside shell; at least one first non-MRI diagnosing means (1NMRI such as one device selected from the following X-ray, CT, PET, or an ultra sound device) locatable within said at least one first opening facing said proximal aperture towards said open bore; and optionally at least one second non-MRI diagnosing (2NMRI, such as another device selected from the following X-ray, CT, PET, or an ultra sound device) locatable within said at least one second opening facing said proximal aperture towards said open bore.

It is acknowledged in this respect that the MRD defined above is a commercially available RF shielded and magnetic shielded apparatus by Aspect Imaging Ltd (US).

External beam radiotherapy or teletherapy is the most common form of radiotherapy. The patient sits or lies on a couch and an external source of radiation is pointed at a particular part of the body. In contrast to internal radiotherapy (brachytherapy), in which the radiation source is inside the body, external beam radiotherapy directs the radiation at the tumor from outside the body. Kilovoltage (“superficial”) X-rays are used for treating skin cancer and superficial structures. Megavoltage (“deep”) X-rays are used to treat deep-seated tumors (e.g. bladder, bowel, prostate, lung, or brain). While X-ray and electron beams are by far the most widely used sources for external beam radiotherapy, a small number of centers operate experimental and pilot programs employing heavier particle beams, particularly proton sources. Conventionally, the energy of diagnostic and therapeutic gamma- and X-rays is expressed in kilovolts or megavolts (kV or MV), whilst the energy of therapeutic electrons is expressed in terms of megaelectronvolts (MeV). In the first case, this voltage is the maximum electric potential used by a linear accelerator to produce the photon beam. The beam is made up of a spectrum of energies: the maximum energy is approximately equal to the beam's maximum electric potential times the electron charge. Thus a 1 MV beam will produce photons of no more than about 1 MeV. The mean X-ray energy is only about ⅓ of the maximum energy. Beam quality and hardness may be improved by special filters, which improve the homogeneity of the X-ray spectrum. In the medical field, useful X-rays are produced when electrons are accelerated to a high energy. Some examples of X-ray energies used in medicine are: diagnostic X-rays—20 to 150 kV; superficial X-rays—50 to 200 kV; orthovoltage X-rays—200 to 500 kV; supervoltage X-rays—500 to 1000 kV; megavoltage X-rays—1 to 25 MV. Of these energy ranges, megavoltage X-rays are by far most common in radiotherapy. Orthovoltage X-rays do have limited applications, and the other energy ranges are not typically used clinically. Medically useful photon beams can also be derived from a radioactive source such as iridium-192, caesium-137 or radium-226 (which is no longer used clinically), or cobalt-60. Such photon beams, derived from radioactive decay, are more or less monochromatic and are properly termed gamma rays. The usual energy range is between 300 keV to 1.5 MeV, and is specific to the isotope.

Therapeutic radiation is mainly generated in the radiotherapy department using the following equipment: Orthovoltage units. These are also known as “deep” and “superficial” machines depending on their energy range. Orthovoltage units have essentially the same design as diagnostic X-ray machines. These machines are generally limited to less than 600 kV. Linear accelerators (“linacs”) which produce megavoltage X-rays. The first use of a linac for medical radiotherapy was in 1953 (see also radiotherapy). Commercially available medical linacs produce X-rays and electrons with an energy range from 4 MeV up to around 25 MeV. The X-rays themselves are produced by the rapid deceleration of electrons in a target material, typically a tungsten alloy, which produces an X-ray spectrum via bremsstrahlung radiation. The shape and intensity of the beam produced by a linac may be modified or collimated by a variety of means. Thus, conventional, conformal, intensity-modulated, tomographic, and stereotactic radiotherapy are all produced by specially-modified linear accelerators. Cobalt units which produce stable, dichromatic beams of 1.17 and 1.33 MeV, resulting in an average beam energy of 1.25 MeV. The role of the cobalt unit has partly been replaced by the linear accelerator, which can generate higher energy radiation. Cobalt treatment still has a useful role to play in certain applications (for example the Gamma Knife) and is still in widespread use worldwide, since the machinery is relatively reliable and simple to maintain compared to the modern linear accelerator.

X-rays are generated by bombarding a high atomic number material with electrons. If the target is removed (and the beam current decreased) a high energy electron beam is obtained. Electron beams are useful for treating superficial lesions because the maximum of dose deposition occurs near the surface. The dose then decreases rapidly with depth, sparing underlying tissue. Electron beams usually have nominal energies in the range 4-20 MeV. Depending on the energy this translates to a treatment range of approximately 1-5 cm (in water-equivalent tissue). Energies above 18 MeV are used very rarely. Although the X-ray target is removed in electron mode, the beam must be fanned out by sets of thin scattering foils in order to achieve flat and symmetric dose profiles in the treated tissue. Hadron therapy involves the therapeutic use of protons, neutrons, and heavier ions (fully ionized atomic nuclei). Of these, proton therapy is by far the most common, though still quite rare compared to other forms of external beam radiotherapy.

A typical multi-leaf collimator (MLC) consists of 2 sets of 40-80 leaves, each around 5 mm to 10 mm thick and several cm in the other two dimensions. Newer MLCs now have up to 160 leaves. Each leaf in the MLC is aligned parallel to the radiation field and can be moved independently to block part of the field. This allows the dosimeters to match the radiation field to the shape of the tumor (by adjusting the position of the leaves), thus minimizing the amount of healthy tissue being exposed to radiation. On a machine without an MLC this must be accomplished using several hand-crafted blocks.

Intensity modulated radiation therapy (IMRT) is an advanced radiotherapy technique used to minimize the amount of normal tissue being irradiated in the treatment field. In some systems this intensity modulation is achieved by moving the leaves in the MLC during the course of treatment, thereby delivering a radiation field with a non-uniform (i.e. modulated) intensity. With IMRT, radiation oncologists are able to break up the radiation beam into many “beamlets.” This allows radiation oncologists to vary the intensity of each beamlet. With IMRT, doctors are often able to further limit the amount of radiation received by healthy tissue near the tumor. Doctors have found this sometimes allowed them to safely give a higher dose of radiation to the tumor, potentially increasing the chance of a cure.

Image-guided radiation therapy (IGRT) augments radiotherapy with imaging to increase the accuracy and precision of target localization, thereby reducing the amount of healthy tissue in the treatment field. The more advanced the treatment techniques become in terms of dose deposition accuracy, the higher become the requirements for IGRT. In order to allow patients to benefit from sophisticated treatment techniques as IMRT or Hadron Therapy, patient alignment accuracies of 0.5 mm and less become desirable. Therefore, new methods like stereoscopic digital kilovoltage imaging based patient position verification (PPVS) to alignment estimation based on in-situ Cone-Beam CT enrich the range of modem IGRT approaches.

Reference is thus made to FIGS. 7a and 7b schematically illustrating a dual modality X-ray/MRI device for image-guided radiation therapy of a patient. In FIG. 7a, two X-ray collimators (420, 410 for example) are either temporarily or not affixed within opening at opposite directions at each side wall of the device. Collimated X-rays (422 and 411, respectively) are emitted over an object, here e.g., patient head (1) having a tumor to analyze and treat. 422 and 411 are emitted in parallel yet opposite directions. In FIG. 7b, however, the two X-ray collimators (420, 410 for example) are either temporarily or not affixed within opening at opposite directions at each side wall of the device. Collimated X-rays (422 and 411, respectively) are emitted over the object, here again, e.g., patient head (1) having a tumor to analyze and treat. 422 and 411 are converges at a predefined angle.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A two step method of diagnosing a medical condition in a patient, said method comprising steps of:
(a) immobilizing (301) a patient (2) or a portion thereof (1) to a support (300); performing steps selected from a group consisting of:
(i) (b) accommodating said immobilized patient (2) or said immobilized portion (1) thereof in a CT device (200) and scanning the same; and,
(c) placing said immobilized patient (2) or said immobilized portion (1) thereof within an MRI device (MRD, 100) and imaging the same such that, because of said immobilization, the relationship between orientation of said patient (2) or said portion (1) thereof during said CT scanning and orientation of said patient (2) or said portion (1) thereof during said MRI imaging is selected from a group consisting of: said orientations are substantially identical, said relationship is 2D spatially retrievable, said relationship is 3D spatially retrievable and any combination thereof; and,
(ii) (b) placing said immobilized patient (2) or said immobilized portion (1) thereof within an MRI device (MRD, 100) and imaging the same; and,
(c) accommodating said immobilized patient (2) or said immobilized portion (1) thereof in a CT device (200) and scanning the same such that, because of said immobilization, the relationship between orientation of said patient (2) or said portion (1) thereof during said CT scanning and orientation of said (2) or said portion (1) thereof during said MRI imaging is selected from a group consisting of: said orientations are substantially identical, said relationship is 2D spatially retrievable, said relationship is 3D spatially retrievable and any combination thereof;
(d) forming at least one combined image by a means selected from a group consisting of: superimposing at least a portion of at least one said CT scan and at least a portion of at least one said MRI image, correlating between at least a portion of at least one said CT scan and at least a portion of at least one said MRI image and any combination thereof; and
(e) analyzing said combined image thereby diagnosing said medical condition of said patient (2) or said portion (1) thereof.

2. The two-step method of claim 1, additionally comprising step of selecting said portion of said patient from a group consisting of: head, liver, pancreas, kidney, gall bladder, thorax, limb and any combination thereof.

3. The two-step method of claim 1, additionally comprising step of selecting said support from a group consisting of: a movable gantry (302), a movable hospital bed, and any combination thereof.

4. The two-step method of claim 1, additionally comprising steps of providing said movable gantry (302) with at least two configurations, at least one first configuration for accommodating said patient (2) or said portion (1) thereof in a CT device (200) and at least one second configuration for placing said patient (2) or said portion (1) thereof within an MRI device (MRD, 100); and providing means for transferring said gantry from said at least one first configuration to said at least one said second configuration.

5. The two-step method of claim 4, additionally comprising step of selecting said means for transferring said gantry (302) from said at least one first configuration to said at least one said second configuration from a group consisting of: a swiveling mechanism, a rotating mechanism, a translating mechanism, a reciprocating mechanism and any combination thereof

6. A single step method of diagnosing an object; said method comprising steps of:
(a) providing an open-bore MRI device (MRD, 100) with at least one opening (103) having a proximal aperture within said MRI open bore (e.g., field of view) and a distal aperture at said MRD outside shell;
(b) providing at least one non-MRI diagnosing (NMRI) effector within said at least one opening facing said proximal aperture towards said open bore;
(c) accommodating said object (2) or a portion (1) thereof within said open bore (the FOV);
(d) NMRI imaging said object (2) or said portion (1) thereof;
(e) MR imaging said object (2) or said portion (1) thereof, said MR imaging occurring at a time selected from a group consisting of: before at least one said NMRI imaging, during at least one said NMRI imaging, after at least one said NMRI imaging and any combination thereof;
(f) forming at least one combined image by a means selected from a group consisting of: superimposing at least a portion of at least one said NMRI image and at least a portion of at least one said MR image, correlating between at least a portion of at least one said NMRI image and at least a portion of at least one said MR image, and any combination thereof; and
(g) analyzing said combined image
thereby diagnosing said object (2) or said portion (1) thereof.

7. The method of claim 6, wherein said object is selected from a group consisting of: adult humans, human children, neonates, premature babies, laboratory animals and portions thereof, histology preparations and plants.

8. The method of claim 7, wherein said diagnosing is diagnosing of a medical condition.

9. The method of claim 8, wherein said medical condition is selected from a group consisting of: neoplasm, vascular disease, posterior cranial fossa lesions, cervicomedullary lesions, or intracranial pressure disorders, hemorrhage, cerebrovascular accident, traumatic brain injury and any combination thereof.

10. The method of claim 7, wherein said portion of said object is selected from a group consisting of: head, liver, pancreas, kidney, gall bladder, thorax, limb and any combination thereof.

11. The method of claim 6, wherein said at least one NMRI effector is selected from a group consisting of: an X-ray device, a CT scanner, a PET device, an ultra sound device and any combination thereof.

12. A multiple modality open-bore MRI device for diagnosing an object comprising:
(a) an open-bore MRI device (MRD, 100) with at least one opening (103), each said at least one opening characterized by a proximal aperture within said MRI open bore (e.g., field of view, FOV) and a distal aperture at said MRD outside shell;
(b) at least one non-MRI diagnosing (NMRI) effector within said at least one opening facing said proximal aperture towards said open bore

13. The multiple modality open-bore MRI device for diagnosing an object; wherein said MRI is operative in a method comprising steps of:
(a) accommodating said object within said open bore (the FOV);
(b) NMRI imaging said object;
(c) MR imaging said object, said MR imaging occurring at a time selected from a group consisting of: before at least one said NMRI imaging, during at least one said NMRI imaging, after at least one said NMRI imaging and any combination thereof;
(d) forming at least one combined image by a means selected from a group consisting of: superimposing at least a portion of at least one said NMRI image and at least a portion of at least one said MR image, correlating between at least a portion of at least one said NMRI image and at least a portion of at least one said MR image, and any combination thereof; and
(e) analyzing said combined image.

14. The device of claim 12, wherein said object is selected from a group consisting of: adult humans, human children, neonates, premature babies, laboratory animals and portions thereof, histology preparations and plants.

15. The device of claim 14, wherein said diagnosing is diagnosing of a medical condition.

16. The device of claim 15, wherein said medical condition is selected from a group consisting of: neoplasm, vascular disease, posterior cranial fossa lesions, cervicomedullary lesions, or intracranial pressure disorders, hemorrhage, cerebrovascular accident, traumatic brain injury and any combination thereof.

17. The device of claim 14, wherein said portion of said object is selected from a group consisting of: head, liver, pancreas, kidney, gall bladder, thorax, limb and any combination thereof.

18. The device of claim 12, wherein said at least one NMRI effector is selected from a group consisting of: an X-ray device, a CT scanner, a PET device, an ultra sound device and any combination thereof.

19. The device of claim 12, adapted for image-guided radiation therapy.
